(19) United States
(12) Patent Application Publication
(10) Pub. No.: US 2024/0041484 A1
(43) Pub. Date: Feb. 8, 2024

(54) EXTENDED CATHETER AND CATHETER SYSTEM FOR REMOVING EMBOLUS IN SMALL BLOOD VESSEL

(71) Applicant: BROSMED MEDICAL CO., LTD., Dongguan (CN)

(72) Inventors: Zhijun Zhang, Dongguan (CN); Chaosheng Liu, Dongguan (CN); Guansheng Zhong, Dongguan (CN)

(21) Appl. No.: 18/484,378

(22) Filed: Oct. 10, 2023

(30) Foreign Application Priority Data
Apr. 20, 2023 (CN) 202310430529.6

(51) Int. Cl. A61B 17/22

(52) U.S. Cl. CPC A61B 17/22032; A61B 2017/22069; A61B 2017/22038; A61B 2017/22079; A61M 25/1011

(57) ABSTRACT

An extended catheter includes a catheter body, a pushing reinforcement wire, a pushing rod, and a positioning balloon. The catheter body is provided with a delivery channel, and a proximal end of the catheter body is provided with an inlet connected to the delivery channel. The pushing reinforcement wire is arranged on a side wall of the catheter body along an axial direction of the catheter body. A distal end of the pushing rod is connected to the proximal end of the catheter body, and an interior of the pushing rod is provided with a filling channel. The positioning balloon is sleeved on the catheter body, and an inner cavity of the positioning balloon is connected to the filling channel. Problem of existing catheters unable to effectively reach the designated position in curved and narrow branch blood vessels for treatment is solved.

### FIELD OF THE INVENTION

The invention relates to the technology field of medical apparatus and instruments, in particular to an extended catheter and a catheter system for removing an embolus in a small blood vessel.

### BACKGROUND OF THE INVENTION

Percutaneous coronary intervention (PCI) refers to the technique of using percutaneous puncture technology to deliver a balloon catheter or other related instruments into the coronary arteries to relieve stenosis or obstruction and reconstruct coronary blood flow. With the continuous development of medicine, medical interventional surgery has also made great progress.

Minimally invasive interventional therapy for diseases such as the heart and blood vessels is widely practiced by inserting catheters into blood vessels. In interventional therapy, a guiding catheter is used to guide the catheter device (such as a balloon catheter, a stent delivery catheter, etc.) for treatment to the lesion, and support the delivery of the catheter device through the reaction force generated by pressure on the blood vessel wall. However, a single guiding catheter cannot provide sufficient back support, and in some cases, the catheter device cannot pass through the stenosis and reach the lesion. Therefore, in addition to guiding the catheter, an extended catheter should also be used to obtain additional back support. However, when using existing extended catheters for treatment in curved and narrow branch blood vessels, there may be insufficient pushing force due to the longer insertion path, resulting in the inability of the extended catheters to effectively reach the designated location for treatment in the curved and narrow branch blood vessels.

Furthermore, when placing an extended catheter in the lumen of the guiding catheter, there may be a gap between the extended catheter and the lumen of the guiding catheter, causing the extended catheter to slip or even dislodge relative to the guiding catheter, which not only prolongs the surgical time, but also causes a significant risk to the patient.

In addition, for an embolus in a small blood vessel, such as cerebral hemorrhage, there are mainly conservative drug treatment and surgical treatment. T-PA (tissue type plasminogen activator, an anticoagulant) can be used for thrombolysis, which can effectively inhibit the spread of thrombus, and accelerate thrombus dissolution and vascular recanalization. However, systemic t-PA treatment may increase the risk of cerebral hemorrhage and other hemorrhagic complications. Patients receiving t-PA treatment are more likely to experience symptomatic cerebral hemorrhage within the first 36 hours of treatment. The frequency of symptomatic bleeding increases when t-PA is used more than 3 hours after a stroke. In addition to the time restrictions for using t-PA in acute ischemic stroke, other contraindications include: if the patient has had a stroke or severe head trauma in the past 3 months, if the patient's systolic blood pressure is above 185 mmHg or diastolic blood pressure is above 110 mmHg, if the patient needs active treatment to reduce blood pressure to a specified limit, if the patient is taking anticoagulants or has a bleeding tendency, and/or if the patient has recently undergone invasive surgery. Therefore, only a small portion of selected stroke patients are eligible to receive t-PA.

For many years, mechanical removal has been used for embolic substances in various parts of the vascular system. Mechanical therapy includes capturing and removing clots, dissolving clots, destroying and aspirating clots, and/or forming flow channels through the clots. However, it is still difficult to completely remove all blood clots. Furthermore, due to the small size and numerous branches of cerebral blood vessels, it is a challenge to introduce mechanical devices for capturing and removing clots or disrupting and aspirating clots into the target area. Even if existing extended catheters are used to mechanically intervene at the site of the emboli, it is easy to cause damage to the blood vessels due to the metal structure of the existing thrombus fragmenting devices, which may cause significant harm to the patient.

Therefore, it is necessary to provide an extended catheter with sufficient pushing force to solve the problem of the existing extended catheter which is unable to effectively reach the designated position in curved and narrow branch blood vessels for treatment, as well as to provide a catheter system with the extended catheter for removing an embolus in a small blood vessel.

### SUMMARY OF THE INVENTION

A first objective of the invention is to provide an extended catheter that can ensure sufficient pushing force, solve the problem of existing extended catheters being unable to effectively reach the designated position in curved and narrow branch blood vessels for treatment.

A second objective of the invention is to provide a catheter system for removing an embolus in a small blood vessel, which has an extended catheter to solve the problem of the existing catheter being unable to effectively reach the designated position in curved and narrow branch blood vessels for treatment.

To achieve the first objective mentioned above, the invention provides an extended catheter, including a catheter body, a pushing reinforcement wire, a pushing rod and a positioning balloon. The catheter body is provided with a delivery channel for transporting medical devices, and a proximal end of the catheter body is provided with an inlet connected to the delivery channel; the pushing reinforcement wire is arranged on the side wall of the catheter body along the axial direction of the catheter body; the distal end of the pushing rod is connected to the proximal end of the catheter body, and the interior of the pushing rod is provided with a filling channel; the positioning balloon is sleeved on the catheter body and located near the proximal end of the catheter body, and the inner cavity of the positioning balloon is connected to the filling channel.

Compared with the prior art, the extended catheter of the invention is provided with a pushing reinforcement wire in the axial direction of the catheter body, which makes the pushing rod less prone to bending when pushing the catheter body into curved and narrow branch blood vessels, thereby improving the pushing force of the extended catheter and facilitating the extension of the catheter into curved and narrow branch blood vessels to solve the problem of existing catheters unable to effectively reach the designated position in curved and narrow branch blood vessels for treatment. The extended catheter of the invention is also provided with a positioning balloon at the proximal end of the catheter body, and a filling channel is set inside the pushing rod, the positioning balloon can be expanded through the filling channel, so that the outer wall of the positioning balloon after expansion tightly adheres to the inner cavity of the guiding catheter, making it difficult for the extended catheter to slip or dislodge within the guiding catheter. In such a way, the axial stability of the extended catheter in the guiding catheter can be enhanced, and the structure is simple and convenient to use, which is conducive to large-scale promotion and application.

Preferably, the proximal end of the pushing reinforcement wire is connected to the distal end of the pushing rod.

Preferably, the extended catheter also includes a connector configured at the proximal end of the catheter body. The connector includes a connecting piece and a plurality of connecting strips in an arc shaped structure. The connecting strips are connected on opposite sides of the connecting piece, and the pushing rod is connected to the connecting piece. The central axis of each connecting strip is the same as the central axis of the catheter body.

Preferably, the catheter body has a hardness decreasing gradually from the proximal end of the catheter body to a distal end thereof.

Preferably, the pushing reinforcement wire has a hardness decreasing gradually from a proximal end to a distal end thereof.

To achieve the second purpose mentioned above, the invention provides a catheter system for removing an embolus in a small blood vessel, including a guiding catheter, a guiding wire, and the extended catheter. The guiding catheter is provided with a guiding channel, the extended catheter is inserted into the guiding channel, a distal end of the catheter body penetrates from a distal end of the guiding channel, the proximal end of the catheter body is located within the guiding channel, the proximal end of the pushing rod is located on an outer side of a proximal end of the guiding channel, the delivery channel is connected with the guiding channel via the inlet, the positioning balloon is configured to press against an inner wall of the guiding channel after expansion, the guiding wire is arranged to move through the guiding channel and the delivery channel, a distal end of the guiding wire is exposed from a distal end of the delivery channel, and the distal end of the guiding wire is provided with a thrombus fragmenting device.

Compared with the prior art, the catheter system for removing an embolus in a small blood vessel in the invention has an extended catheter, and a pushing reinforcement wire is provided in the axial direction of the catheter body of the extended catheter, which makes the pushing rod less prone to bending when pushing the catheter body into curved and narrow branch blood vessels, thereby increasing the pushing force of the extended catheter and facilitating the extension of the catheter into curved and narrow branch blood vessels to solve the problems of existing catheters as mentioned in the background. The proximal end of catheter body is located at the distal end of guiding catheter, after the expansion of positioning balloon, the outer wall of positioning balloon is tightly adhered to the guiding channel of guiding catheter to block the gap between guiding catheter and catheter body. Further, the guiding catheter is connected to the delivery channel, and the contrast agent/drug is injected from the proximal end of guiding catheter, and flows along the guiding channel a and enter the delivery channel through the inlet for super-selective angiography (by injecting contrast agent to display the pathological changes of the coronary artery) or administration. In addition, as the delivery channel is connected to the guiding channel, the proximal end of the guiding catheter can be connected to an external aspiration device to aspirate the thrombus and plaques, which can avoid the frequent replacement of intervention catheters during surgery, shorten surgical time, reduce surgical difficulty, and reduce patient's pains. When the guiding catheter cannot penetrate into the distal end of a small blood vessel to reach the clot or other desired target site, the extended catheter can be introduced into the guiding catheter and extended beyond the distal end of the guiding catheter, thereby expanding the range of the aspiration system. When the size of the thrombus is too big to be aspirated into the extended catheter, the thrombus fragmenting device of the guiding wire may act on the thrombus for thrombus fragmentation first, and then the thrombus fragmentations may be aspirated into the extended catheter.

Preferably, the thrombus fragmenting device includes a first thrombus fragmenting balloon and a second thrombus fragmenting balloon, a volume size of the first thrombus fragmenting balloon is larger than that of the second thrombus fragmenting balloon, and the first thrombus fragmenting balloon and second thrombus fragmenting balloon are arranged alternately along an axial direction of the guiding wire to form an axial serrated structure.

Preferably, the first thrombus fragmenting balloon and second broken thrombus fragmenting balloon are arranged in an alternating manner along the circumferential direction of the guiding wire and form a circumferential serrated structure.

Preferably, the guiding wire is provided with a flow channel, and both the first thrombus fragmenting balloon and second thrombus fragmenting balloon are connected to a distal end of the flow channel.

Preferably, the proximal end of the guiding wire is provided with a guiding wire needle holder, and the guiding needle holder is connected to a proximal end of the flow channel.

Preferably, the distal end of the guiding catheter is provided with a Y-shaped connecting valve, the Y-shaped connecting valve has a first interface and a second interface respectively connected to the guiding channel, the guiding wire and the extended catheter are both inserted into the first interface, and the proximal end of the pushing rod is located on an outer side of the Y-shaped connecting valve.

Preferably, the catheter system for removing an embolus in a small blood vessel also includes an aspiration device for aspirating thrombus, which is connected to the first interface and connected to the guiding channel.

### BRIEF DESCRIPTION OF THE DRAWINGS

The accompanying drawings facilitate an understanding of the various embodiments of the invention. In such drawings:

FIG. 1 is a structural diagram of the extended catheter according to one embodiment of the invention;

FIG. 2 is a detailed diagram of the connection between the pushing rod and the catheter body of the extended catheter according to one embodiment of the invention;

FIG. 3 is a detailed view of the catheter body of the extended catheter according to one embodiment of the invention at the inlet position;

FIG. 4 is an enlarged view of portion A in FIG. 3;

FIG. 5 is a cross-sectional view of the catheter body of the extended catheter according to one embodiment of the invention;

FIG. 6 is a structural diagram of the guiding catheter of the catheter system according to one embodiment of the invention;

FIG. 7 is a structural diagram of the connection between the extended catheter and the guiding catheter according to one embodiment of the invention;

FIG. 8 is a structural diagram of the guiding wire of the catheter system according to one embodiment of the invention;

FIG. 9 is a structural diagram showing that the guiding catheter of the catheter system according to one embodiment of the invention is extended into the aortic arch;

FIG. 10 is a structural diagram showing that the catheter system according to one embodiment of the invention reaches to the narrow right internal carotid artery by the extended catheter;

FIG. 11 is a structural diagram showing that the guiding wire of the catheter system according to one embodiment of the invention enters the thrombus;

FIG. 12 is a detailed view showing that the guiding wire of the catheter system according to one embodiment of the invention enters the thrombus;

FIG. 13 is a structural diagram showing thrombus fragmentation by the guiding wire of the catheter system according to one embodiment of the invention; and

FIG. 14 shows the flow direction of the thrombus fragmentations when aspirated.

### DETAILED DESCRIPTION OF ILLUSTRATED EMBODIMENTS

In order to provide a detailed explanation of the technical content and structural features of the invention, the following will be further explained in conjunction with the embodiments and accompanying drawings.

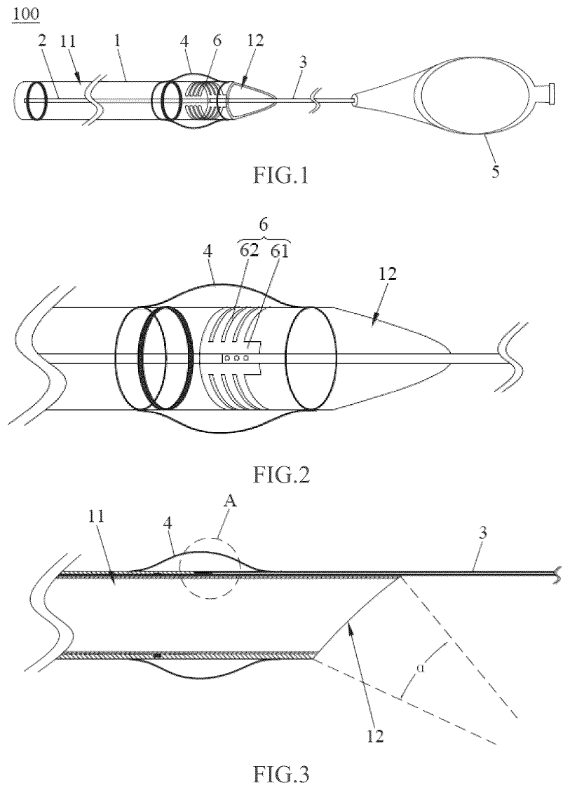

Referring to FIGS. 1 to 8, the catheter system 200 for removing an embolus in a small blood vessel according to the invention includes a guiding catheter 201, a guiding wire 202, and an extended catheter 100. Specifically, the extended catheter 100 includes a catheter body 1, a pushing reinforcement wire 2, a pushing rod 3, and a positioning balloon 4. The catheter body 1 is provided with a delivery channel 11 for transporting medical devices, and the proximal end of the catheter body 1 is provided with an inlet 12 connected to the delivery channel 11. The pushing reinforcement wire 2 is arranged along the axial direction of the catheter body 1 on the side wall of the catheter body 1. The distal end of the pushing rod 3 is connected to the proximal end of the catheter body 1, and the interior of the pushing rod 3 is provided with a filling channel 31. The positioning balloon 4 is set on the catheter body 1 and located near the proximal end of the catheter body 1. The inner cavity of the positioning balloon 4 is connected to the filling channel 31. The guiding catheter 201 is provided with a guiding channel 201a, and the extended catheter 100 is inserted into the guiding channel 201a. The distal end of the catheter body 1 penetrates from the distal end of the guiding channel 201a, the proximal end of the catheter body 1 is located inside the guiding channel 201a, the proximal end of the pushing rod 3 is located outside the proximal end of the guiding channel 201a, and the inlet 12 connects the delivery channel 11 with the guiding channel 201a. The positioning balloon 4 expands is configured to press against an inner wall of the guiding channel 201a, the guiding wire 202 can move through the guiding channel 201a and the delivery channel 11, and the distal end of the guiding wire 202 is exposed from the distal end of the delivery channel 11. The distal end of the guiding wire 202 is equipped with a thrombus fragmenting device 202a for fragmenting the thrombi 300.

Referring to FIG. 8, in this embodiment, the thrombus fragmenting device 202a includes a first thrombus fragmenting balloon 202a1 and a second thrombus fragmenting balloon 202a2, and the volume size of the first thrombus fragmenting balloon 202a1 is larger than that of the second thrombus fragmenting balloon 202a2. The first thrombus fragmenting balloon 202a1 and the second thrombus fragmenting balloon 202a2 are arranged alternately along the axial direction of the guiding wire 202 and form an axial serrated structure. Specifically, the first thrombus fragmenting balloon 202a1 and the second thrombus fragmenting balloon 202a2 are arranged alternately along the circumferential direction of the guiding wire 202 and form a circumferential serrated structure. More specifically, the length of the thrombus fragmenting device 202a ranges from 40 mm to 80 mm, and the diameter ranges from 8 mm to 18 mm. Furthermore, both the first and second thrombus fragmenting balloon 202a1 and 202a2 are semi elliptical in shape, with a working pressure of 6 atm and a burst pressure of 18 atm. However, the structure of the thrombus fragmenting device 202a is not limited by this. For example, the thrombus fragmenting device 202a also may be a spring, a protrusion, and so on.

Referring to FIG. 8, in this embodiment, the guiding wire 202 is provided with a flow channel 202b, and both the first and second thrombus fragmenting balloon 202a1 and 202a2 are connected to the distal end of the flow channel 202b. Furthermore, the proximal end of the guiding wire 202 is provided with a guiding wire needle holder 202c, which is connected to the proximal end of the flow channel 202b.

Referring to FIG. 8, both the proximal and distal ends of the thrombus fragmenting device 202a are provided with a marker point 202a3 for identifying the position. This marker point 202a3 may be an X-ray opaque marker point 202a3, which is not limited however.

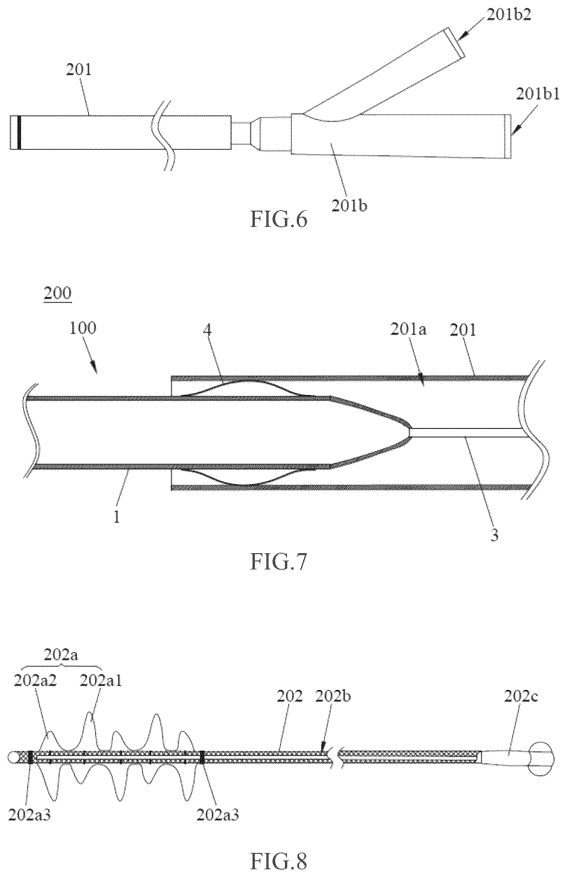

Referring to FIG. 6, the distal end of the guiding catheter 201 is provided with a Y-shaped connecting valve 201b, which has a first interface 201b1 and a second interface 201b2. The first interface 201b1 and the second interface 201b2 are respectively connected to the guiding channel 201a. The guiding wire 202 and the extended catheter 100 are both inserted into the first interface 201b1, and the proximal end of the pushing rod 3 is located on the outer side of the Y-shaped connecting valve 201b. Furthermore, the catheter system 200 of the invention also includes an aspiration device for aspirating thrombi 300, which is connected to the first interface 201b1 and connected to the guiding channel 201a.

Referring to FIGS. 1 and 2, in this embodiment, the proximal end of the pushing reinforcement wire 2 is connected to the distal end of the pushing rod 3, further improving the pushing force of the extended catheter 100. Specifically, the pushing reinforcement wire 2 and the pushing rod 3 are integrated structures, which is not limited however. For example, the pushing reinforcement wire 2 and the pushing rod 3 can also be two components and connected through existing methods such as clamping and locking.

Referring to FIG. 1, the extended catheter 100 also includes a catheter holder 5, which is connected to the proximal end of the pushing rod 3. The catheter holder 5 is provided with a connecting hole connected to the filling channel 31.

Referring to FIG. 5, the catheter body 1 includes a three-layer composite structure consisting of an inner layer 13, an intermediate reinforcement layer 14, and an outer layer 15 arranged sequentially from the inside out, which can provide strong support, pressure resistance, and bending resistance.

In an embodiment, the material of the inner layer 13 is PTEF (polytetrafluoroethylene) or LLDPE (linear low-density polyethylene). PTEF has high lubrication and non-adhesion, making it convenient for other instruments to pass through the delivery channel 11 by the inner layer 13. LLDPE has high softening and melting temperatures, has advantages such as high strength, toughness, rigidity, heat resistance, and cold resistance, and also has good resistance to environmental stress cracking, impact strength, and tear strength.

In an embodiment, the outer layer 15 is made of one or more mixtures of polyether block polyamide, nylon, and polyurethane elastomers, ensuring a smoother appearance and feel on the surface of the catheter body 1, and fully protecting blood vessels from thrombosis, dissection, etc. Furthermore, the soft hardness of the outer layer 15 material decreases sequentially from the proximal end to the distal end of the catheter body 1. This not only avoids deformation, but also makes it easier to pass through the tortuous lesion area. The distal end is made of flexible materials to avoid damage to the blood vessel wall during the pushing process, better meeting the needs of pushing the catheter body 1 into the human blood vessels. This not only allows doctors to operate more accurately and conveniently, but also reduces the pain of patients during the surgery process.

In an embodiment, the intermediate reinforcement layer 14 includes a stainless-steel woven mesh or spring, thereby improving the strong support, pressure resistance, and bending resistance of the extended conduit 100, which is not limited however.

Referring to FIG. 1, in one embodiment, the material of the positioning balloon 4 is one of silicone rubber, polyurethane elastomer, and thermoplastic elastomer. Silicone rubber also has prominent characteristics of physiological inertness and does not cause coagulation, making it widely used in the medical field. Polyurethane resin, as a polymer material with high strength, tear resistance, and wear resistance, is widely used in daily life, industrial and agricultural production, medicine, and other fields. Thermoplastic elastomer TPE/TPR, also known as artificial rubber or synthetic rubber, not only has the excellent properties of high elasticity, aging resistance, and oil resistance of traditional cross-linked vulcanized rubber, but also has the characteristics of convenient processing and wide processing methods of ordinary plastics.

Referring to FIG. 2, in an embodiment, the radial cross-sectional structure of the pushing rod 3 is one of elliptical, circular, and semi arc-shaped. Elliptical and semi curved pushing rods 3 may have reduced thickness and smaller occupied space, which facilitates the passage of other instruments. Circular pushing rods 3 may ensure a better strength and improved push performance.

In an embodiment, the pushing rod 3 is made of a Hypotube, which has high hardness, good anti twisting performance, and better push performance.

Referring to FIG. 2, in an embodiment, the extended catheter 100 also includes a connector 6 located at the proximal end of the catheter body 1. Specifically, the connector 6 connects the intermediate reinforcement layer 14 and the pushing rod 3. The connector 6 is made of metal material, and the connector 6, pushing rod 3, and intermediate reinforcement layer 14 are connected by laser welding to ensure a firm connection between the pushing rod 3 and the catheter body 1 in the middle position of the catheter body 1, which will not cause damage to blood vessels.

Referring to FIG. 2, in an embodiment, the connector 6 includes a connecting piece 61 and multiple connecting strips 62 in an arc-shaped structure. The multiple connecting strips 62 are connected on opposite sides of the connecting piece 61, and the pushing rod 3 is connected to the connecting piece 61. The central axis of each connecting strip 62 is the same as the central axis of the catheter body 1. The connecting piece 61 and each connecting strip 62 is connected to the intermediate reinforcement layer 14, and each connecting strip 62 is wrapped and connected to the inner layer 13. Specifically, the connector 6 may be located in the intermediate layer of catheter body 1, but not limited to this. For example, the connector 6 can also be located in the outer layer of catheter body 1. The connecting strip 62 is wrapped and connected to the outer side of the inner layer 13, ensuring that the connecting piece 6 is securely connected to the catheter body 1.

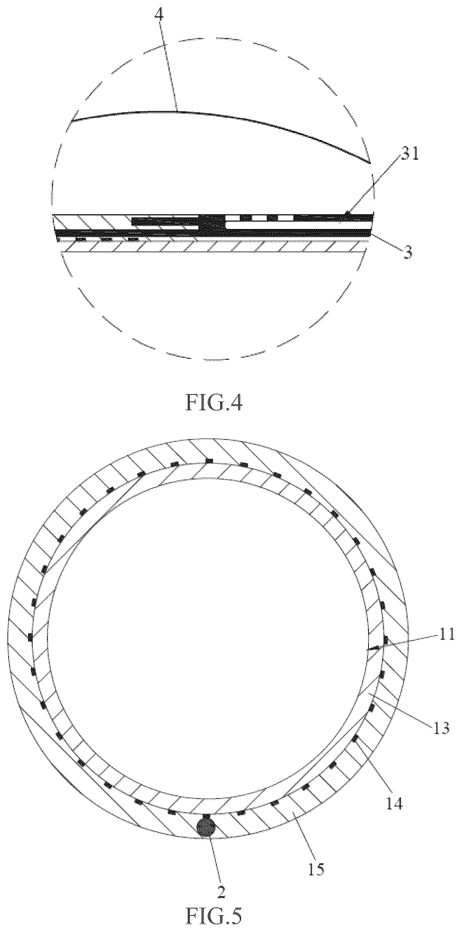

Referring to FIGS. 3 and 4, in an embodiment, the distal end of the pushing rod 3 is provided with a first through hole, which is connected to the filling channel 31. The outer layer 15 is provided with a second through hole, which corresponds to the position of the first through hole. The filling channel 31 is connected to the inner cavity of the positioning balloon 4 through the first through hole. There are multiple first through holes arranged side by side. Due to the first and the second through holes, the positioning balloon 4 and filling channel 3 are guided, which facilitates the operator to inject fluid and other media through the filling channel 31 to fill the positioning balloon 4, to quickly fix the positioning balloon 4 in the guiding channel 201a of the guiding catheter 201, and finally to seal it.

Referring to FIGS. 1 to 3, in an embodiment, the inlet 12 is a tapered structure. The proximal end of the catheter body 1 is inclined and provided with the inlet 12. The cross-sectional area of the inlet 12 is larger than that of the delivery channel 11, making it easier for other instruments to be pushed and transmitted along the inlet 12 to the delivery channel 11. In another embodiment, the inlet 12 may be in the shape of a water droplet, and the transition between the softness and hardness of the connection between the catheter body 1 and the pushing rod 3 is smooth, ensuring a smooth transition of the inlet 12 and improving the passage of the fast exchange type extended catheter within the blood vessel.

The extended catheter 100 satisfies at least one of the following conditions: the hardness of the catheter body 1 is sequentially decreased from the proximal end to the distal end of the catheter body 1; and the hardness of the pushing reinforcement wire 2 is gradually decreased from the proximal end to the distal end. On one hand, the catheter body 1 may be smoothly pushed into the blood vessels; and on the other hand, the harm caused to patients and the pains of patients during the surgery process are reduced.

In an embodiment, the hardness of the catheter body 1 is sequentially decreased from the proximal end to the distal end of catheter body 1, that is, the hardness of the proximal part of catheter body 1 is greater than the distal part of catheter body 1, which better meets the need for catheter body 1 to be pushed into blood vessels, not only allows doctors to operate more accurately and conveniently, but also reduces patient pain during the surgical process. Specifically, the hardness of the catheter body 1 may be set based on material distributions or wall thickness changes. Furthermore, the inner diameter of catheter body 1 may be decreased from the proximal end to the distal end thereof, or remain unchanged.

In an embodiment, the inner diameter of the catheter body 1 remains unchanged from the proximal end of the catheter body 1 to the distal end of the catheter body 1. At this time, the hardness of the catheter body 1 may be adjusted through material distribution.

In an embodiment, the inner diameter of catheter body 1 is decreased from the proximal end to the distal end thereof.

In one embodiment, the inner diameter and outer diameter of the catheter body 1 have the same diameter variation, that is, the inner diameter and outer diameter have the same decreasing trend.

In an embodiment, the inner diameter and outer diameter of the catheter body 1 have different diameter variations, that is, the inner diameter has a diameter variation different from the decreasing trend of the outer diameter.

In an embodiment, the decreasing trend of the inner diameter of catheter body 1 is smaller than the decreasing trend of the outer diameter of catheter body 1, that is, the wall thickness of catheter body 1 is increased from the proximal end of catheter body 1 to the distal end of catheter body 1. In other embodiment, the decreasing trend of the inner diameter of catheter body 1 is greater than the decreasing trend of the outer diameter of catheter body 1, that is, the wall thickness of catheter body 1 decreases from the proximal end of catheter body 1 to the distal end of catheter body 1.

In an embodiment, the material of the distal end (tip) of the catheter body 1 is soft, which is not easy to damage the blood vessels therefore.

In an embodiment, the hardness of the pushing reinforcement wire 2 is gradually decreased from the proximal end to the distal end. Specifically, the hardness of the pushing reinforcement wire 2 can be set through material distribution or through wall thickness changes. The hardness change of the pushing reinforcement wire 2 can be achieved through the structure and method of the hardness change of the catheter body 1 mentioned above.

In one embodiment, the pushing rod 3 is made of metal materials, such as stainless steel, mild steel, nickel containing alloys, or metal polymer composite materials. Furthermore, the pushing rod 3 has flexibility to follow the bending shape of the blood vessel.

In an embodiment, the outer surface of the catheter and pushing rod 3 is provided with an anticoagulant coating. The anticoagulant coating is made of bioactive materials. Specifically, the anticoagulant coating may be a hydrophilic coating of negatively charged/heparin like polymers, a polyethylene glycol layer grafted with anticoagulant conformation, a multi-layer composite layer formed by heparin/dopamine and heparin/collagen, or a magnetic layer composed of magnetic materials. Furthermore, the anticoagulant coating may also be made by surface modification of metal surfaces. Specifically, the metal surfaces may be performed with chemical passivation treatment, such as mixed acid passivation treatment, or physical passivation treatment, such as high-temperature heat treatment passivation.

In an embodiment, the positioning balloon 4 is vacuum flattened and attached to the catheter body 1, and the distal end of the positioning balloon 4 is tightly welded with the outer layer 15 of the catheter body 1 to form a sealing end. The proximal end of the positioning balloon 4 is tightly welded with the outer layer 15 of the catheter body 1 and the distal end of the pushing rod 3. The welding connection between the catheter body 1 and the positioning balloon 4 is smooth at both ends, without obvious bumps, which brings less resistance for entering other channels and smoother pushing.

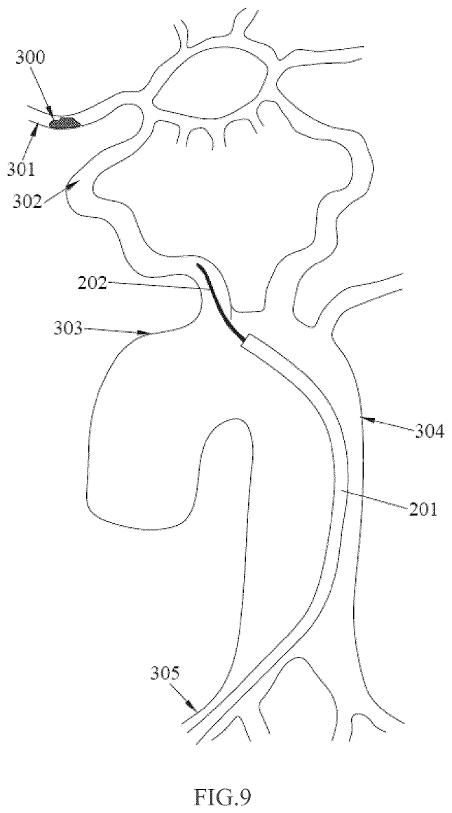

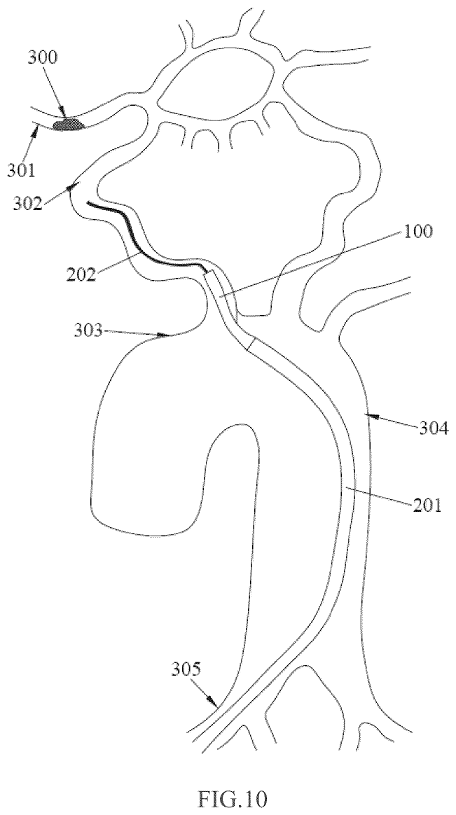

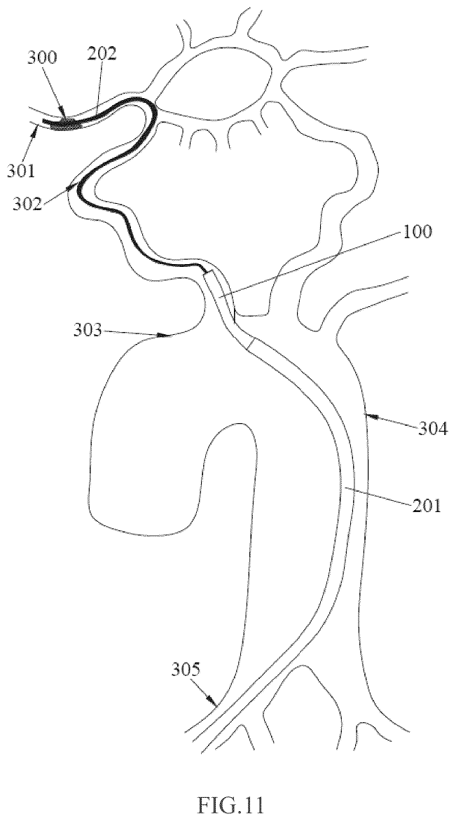

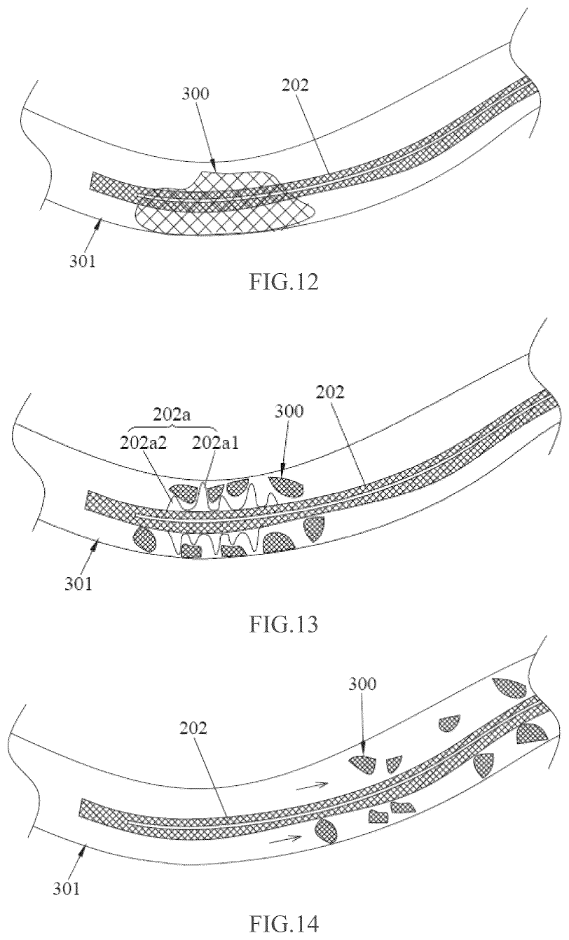

Based on FIGS. 1 to 12, as an example, the catheter system 200 of the invention involves sequentially passing through the femoral artery 305 (inner diameter 8.0±1.4 mm)→aorta 304→aortic arch 303→right internal carotid artery 302→right middle cerebral artery 301 (with an outer diameter of 3-5 mm, an inner diameter of 1-3 mm) for fragmenting the thrombi. The specific working principle of the catheter system 200 is as follows.

Target area entering: the thrombus 300 is located in the right middle cerebral artery 301, the guiding catheter 201 is conveyed to the aorta 304 and the right internal carotid artery 302, and the guiding wire 202 is transported along the proximal end of Y-shaped connecting valve 201b of the guiding catheter 201 to the distal end of the stenosis of the right middle cerebral artery 301 through the right internal carotid artery 302. The extended catheter 100 is pushed along the guiding wire 202, and extended from the Y-shaped connecting valve 201b at the proximal end of the guiding catheter 201 into the guiding catheter 201. The distal end of the catheter body 1 of the extended catheter 100 is exposed from the distal end of the guiding catheter 201, and the proximal end of the catheter body 1 is located in the guiding channel 201a. One end of the pushing rod 3 is located in the guiding channel 201a, and the other end is exposed from the Y-shaped connecting valve 201b, and the catheter holder 5 is located outside the Y-shaped connecting valve 201b. Under the guidance of the guiding wire 202, the catheter body 1 reaches to the designated position. The pushing reinforcement wire 2 exerting force in the catheter body 1 facilitates the guiding catheter 201 to enter the branching and narrow blood vessels.

Transmission channel establishment: the positioning balloon 4 is expanded through the inner cavity of the catheter holder 5 and the filling channel 31 in the pushing rod 3. After expansion, the positioning balloon 4 is anchored in the guiding channel 201a, to block the gap between the guiding catheter 201 and the catheter body 1, and the guiding channel 201a is connected to the delivery channel 11.

Contrast agent/drug delivery: the first interface 201b1 of the pushing rod 3 extending from the Y-shaped connecting valve 201b of the guiding catheter 201 is blocked, and the contrast agent/drug is delivered at the second interface 201b2 of the Y-shaped connecting valve 201b. The contrast agent/drug flows along the guiding channel 201a to the delivery channel 11, then flows out at the distal end of the catheter body 1, and finally flows to the distal end of the coronary artery stenosis for ultra selective contrast/targeted administration.

Thrombus fragmentation: the guiding wire 202 is pushed into the thrombus 300, so that the first and second thrombus fragmenting balloons 202a1 and 202a2 of the thrombus fragmenting device 202a reach to the thrombus 300. Under X-ray fluoroscopy, the position of the serrated balloons formed by the first and second thrombus fragmenting balloons 202a1 and 202a2 are adjusted based on the proximal and distal marking points 202a3 of the thrombus fragmenting device 202a, and then it's determined that the length of the serrated balloons can fully cover the thrombus 300. The pressure pump joint is connected to the guiding wire needle holder 202c, and the developer is injected to fully expand the first and second thrombus fragmenting balloons 202a1 and 202a2 which reach the working pressure. In the parallel direction of the blood vessel, the guiding wire 202 is pushed and retracted, causing the serrated balloon to cut or fragment the thrombus 300 in the horizontal direction, or the guiding wire 202 is rotated at 360° along the circumference of the blood vessel, causing the serrated balloon to cut or fragment the thrombus 300 in the vertical direction of the blood vessel. The simultaneous operations in both horizontal direction and vertical direction bring a quick and efficient thrombus fragmentation. The effect of the thrombus fragmentation for the thrombus 300 may be determined through angiography, to ensure a complete thrombus fragmentation without residue.

Thrombus aspiration by an aspiration device: the first interface 201b1 of the Y-shaped connecting valve 201b of the guiding catheter 201 that extends out of the pushing rod 3 is blocked, an aspiration device is connected to the second interface 201b2 of the Y-shaped connecting valve 201b for aspirating the thrombus 300 or the plaque at the distal end of the catheter body 1. In such a way, the thrombus 300 or the calcified plaques may flow back to guiding channel 201a along delivery channel 11 under an action of negative pressure, and finally aspirated out of the body.

Specifically, when the serrated balloons fragment the thrombus 300, the aspiration may be carried out simultaneously with the fragmentation. Optionally, the aspiration may be carried out after the fragmentation; or it's also feasible to carry out the aspiration first, then the fragmentation, and then the aspiration again. For example, the aspiration may be directly carried out, when the thrombus 300 is small; the aspiration may follow the fragmentation, when the thrombus 300 is large; or the aspiration and the fragmentation may be carried out alternatively, when the thrombus 300 has a long size along the length of the blood vessel. The aspiration may be in a constant mode or a pulse mode. The applied pulse may be selected between positive vacuum and zero vacuum, or between the first lower negative pressure and the second higher negative pressure. Optionally, slight positive and negative pressures may be used alternately.

Guiding wire 202 (or extended catheter 100) withdrawal: a pressure pump is used to pump back the developer from the first and second thrombus fragmenting balloon 202a1 and 202a2 under negative pressure. Under X-ray fluoroscopy, it's determined that the first and second thrombus fragmenting balloon 202a1 and 202a2 are completely deflated without residual developer, thus the guiding wire 202 (or extended catheter 100) is retracted as a whole to exit the body.

In summary, the catheter system 200 of the invention has an extended catheter 100, and a pushing reinforcement wire 2 is provided in the axial direction of the catheter body 1 of the extended catheter 100, which makes the pushing rod 3 less prone to bending when pushing the catheter body 1 into curved and narrow branch blood vessels, thereby increasing the pushing force of the extended catheter 100 and facilitating the extension of the catheter 100 into curved and narrow branch blood vessels to solve the problems of existing catheters as mentioned in the background. The proximal end of catheter body 1 is located at the distal end of guiding catheter 201, after the expansion of positioning balloon 4, the outer wall of positioning balloon 4 is tightly adhered to the guiding channel 201a of guiding catheter 201 to block the gap between guiding catheter 201 and catheter body 1. Further, the guiding catheter 201 is connected to the delivery channel 11, and the contrast agent/drug is injected from the proximal end of guiding catheter 201, and flows along the guiding channel 201a and enter the delivery channel 11 through the inlet 12 for super-selective angiography (by injecting contrast agent to display the pathological changes of the coronary artery) or administration. In addition, as the delivery channel 11 is connected to the guiding channel 201a, the proximal end of the guiding catheter 201 can be connected to an external aspiration device to aspirate the thrombus 300 and plaques, which can avoid the frequent replacement of intervention catheters during surgery, shorten surgical time, reduce surgical difficulty, and reduce patient's pains. When the guiding catheter 201 cannot penetrate into the distal end of a small blood vessel to reach the clot or other desired target site, the extended catheter 100 can be introduced into the guiding catheter 201 and extended beyond the distal end of the guiding catheter 201, thereby expanding the range of the aspiration system. When the size of the thrombus 300 is too big to be aspirated into the extended catheter 100, the thrombus fragmenting device 202a of the guiding wire 202 may act on the thrombus 300 for thrombus fragmentation first, and then the thrombus fragmentations may be aspirated into the extended catheter 100.

The above disclosure is only a preferred example of the invention and cannot be used to limit the scope of present invention. Therefore, any equivalent changes made according to the claims of the invention belong to the scope of the invention.

What is claimed is:

1. An extended catheter, comprising:
a catheter body, provided with a delivery channel for transporting a medical device, and a proximal end of the catheter body being provided with an inlet connected to the delivery channel;
a pushing reinforcement wire, arranged on a side wall of the catheter body along an axial direction of the catheter body;
a pushing rod, a distal end of the pushing rod being connected to the proximal end of the catheter body, and an interior of the pushing rod being provided with a filling channel; and
a positioning balloon, sleeved on the catheter body and located near the proximal end of the catheter body, and an inner cavity of the positioning balloon being connected to the filling channel.

2. The extended catheter according to claim 1, wherein a proximal end of the pushing reinforcement wire is connected to the distal end of the pushing rod.

3. The extended catheter according to claim 1, further comprising a connector configured at the proximal end of the catheter body, wherein the connector comprises a connecting piece and a plurality of connecting strips in an arc shaped structure, the connecting strips are connected on opposite sides of the connecting piece, the pushing rod is connected to the connecting piece, and a central axis of each connecting strip is same as a central axis of the catheter body.

4. The extended catheter according to claim 1, wherein the catheter body has a hardness decreasing sequentially from the proximal end of the catheter body to a distal end thereof.

5. The extended catheter according to claim 1, wherein the pushing reinforcement wire has a hardness decreasing gradually from a proximal end to a distal end thereof.

6. A catheter system for removing an embolus in a small blood vessel, comprising a guiding catheter, a guiding wire, and the extended catheter according to claim 1, wherein the guiding catheter is provided with a guiding channel, the extended catheter is inserted into the guiding channel, a distal end of the catheter body penetrates from a distal end of the guiding channel, the proximal end of the catheter body is located within the guiding channel, the proximal end of the pushing rod is located on an outer side of a proximal end of the guiding channel, the delivery channel is connected with the guiding channel via the inlet, the positioning balloon is configured to press against an inner wall of the guiding channel after expansion, the guiding wire is configured to move through the guiding channel and the delivery channel, a distal end of the guiding wire is exposed from a distal end of the delivery channel, and the distal end of the guiding wire is provided with a thrombus fragmenting device.

7. The catheter system according to claim 6, wherein the thrombus fragmenting device includes a first thrombus fragmenting balloon and a second thrombus fragmenting balloon, a volume size of the first thrombus fragmenting balloon is larger than that of the second thrombus fragmenting balloon, and the first thrombus fragmenting balloon and the second thrombus fragmenting balloon are arranged alternately along an axial direction of the guiding wire to form an axial serrated structure.

8. The catheter system according to claim 7, wherein the first thrombus fragmenting balloon and the second thrombus fragmenting balloon are arranged alternately along a circumferential direction of the guiding wire to form a circumferential serrated structure.

9. The catheter system according to claim 7, wherein the guiding wire is provided with a flow channel, and the first thrombus fragmenting balloon and the second thrombus fragmenting balloon are connected to a distal end of the flow channel.

10. The catheter system according to claim 9, wherein the proximal end of the guiding wire is provided with a guiding wire needle holder, and the guiding wire needle holder is connected to a proximal end of the flow channel.

11. The catheter system according to claim 6, wherein the distal end of the guiding catheter is provided with a Y-shaped connecting valve, the Y-shaped connecting valve has a first interface and a second interface respectively connected to the guiding channel, the guiding wire and the extended catheter are both inserted into the first interface, and the proximal end of the pushing rod is located on an outer side of the Y-shaped connecting valve.

12. The catheter system according to claim 11, further comprising an aspiration device for aspirating thrombi, which is connected to the first interface and connected to the guiding channel.
